(19) United States
(12) Patent Application Publication
(10) Pub. No.: US 2005/0124840 A1
(43) Pub. Date: Jun. 9, 2005

(54) Process for the production of olefins from alkanes with carbon monoxide co-feed and/or recycle

(75) Inventors: Shang Chen, Edmond, OK (US); Steven R. McDonald, Ponca City, OK (US); Zhen Chen, Ponca City, OK (US)

Correspondence Address: DAVID W. WESTPHAL;CONOCOPHILLIPS COMPANY - I.P. Legal, P.O. BOX 1267, PONONCA CITY, OK, 74602-1267, US

(73) Assignee: ConocoPhillips Company, Houston, TX (US)

(21) Appl. No.: 10/730,468

(22) Filed: Dec. 5, 2003

(51) Int. Cl. C07C 5/333

(52) U.S. Cl. 585/658; 585/654

(57) ABSTRACT

A method for producing olefins by oxidative dehydrogenation. In one embodiment, the method comprises feeding a feed to a reactor comprising a catalyst, wherein the feed comprises oxygen, and a carbonaceous material comprising carbon monoxide and a light hydrocarbon; contacting the feed to the catalyst in the reactor; and converting at least a portion of the light hydrocarbon with oxygen to at least one olefin, while simultaneously converting at least a portion of the carbonaceous material with oxygen to carbon dioxide to form a product stream comprising the at least one olefin and by-products. The by-products comprise at least carbon monoxide. In other embodiments, at least a portion of the by-products, which comprise carbon monoxide, is recycled to the reactor. In further embodiments, the light hydrocarbon feed comprises ethane, and the olefin comprises ethylene.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1.Field of the Invention

This invention relates to the field of olefin production and more specifically to the field of oxidative conversion of ethane to produce ethylene.

2.Background of the Invention

There is currently a significant interest in various types of hydrocarbon processing reactions. One such class of reactions involves the chemical conversion of natural gas, a relatively low value reactant, to higher value products. Natural gas comprises several components, including alkanes. Alkanes are saturated hydrocarbons—e.g., compounds consisting of hydrogen (H) and carbon (C)—whose molecules contain carbon atoms linked together by single bonds. The principal alkane in natural gas is methane; however, significant quantities of longer-chain alkanes such as ethane (CH₃CH₃), propane (CH₃CH₂CH₃) and butane (CH₃CH₂CH₂CH₃) are also present. Unlike even longer-chain alkanes, these so-called lower alkanes are gaseous under ambient conditions.

The interest in the chemical conversion of the lower alkanes in natural gas stems from a variety of factors. First, vast reserves of natural gas have been found in remote areas where no local market exists. There is great incentive to exploit these natural gas formations because natural gas is predicted to outlast liquid oil reserves by a significant margin. Unfortunately, though, the transportation costs for the lower alkanes are generally prohibitive, primarily because of the extremely low temperatures needed to liquefy these highly volatile gases for transport. Consequently, there is considerable interest in techniques for converting methane and other gaseous hydrocarbons to higher value and more easily transportable products at the remote site. A second factor driving research into commercial methods for chemical conversion of lower alkanes is their abundant supply at many refineries and the relatively few commercially-viable means of converting them to more valuable products.

Several hydrocarbon processing techniques are currently being investigated for the chemical conversion of lower alkanes. One such technique involves the conversion of methane to higher chain-length alkanes that are liquid or solid at room temperature. This conversion of methane to higher hydrocarbons is typically carried out in two steps. In the first step, methane is converted with an oxidant to produce a mixture of carbon monoxide and hydrogen known as synthesis gas or syngas. In a second step, the syngas is converted to liquid hydrocarbon fuels and solid hydrocarbon waxes using the Fischer-Tropsch synthesis. The high molecular weight waxes thus produced provide an ideal feedstock for hydrocracking, which ultimately yields jet fuel, gasoline, high decane diesel fuel, or blending stocks for such fuels, particularly superior high decane value diesel fuel.

Another important class of hydrocarbon processing reactions relates to the production of olefins from alkanes.-Olefins have traditionally been produced from alkanes by fluid catalytic cracking (FCC) or steam cracking, depending on the size of the alkanes. Heavy olefins are herein defined as containing at least five carbon atoms and are produced by FCC. Light olefins are defined herein as containing two to four carbon atoms and are predominantly produced by steam cracking. Olefins can also be generated from low molecular weight alkanes by dehydrogenation reactions. In a dehydrogenation process, alkanes can be dehydrogenated to produce alkenes.

Alkenes, or olefins, are higher value chemicals than their corresponding alkanes. This is true, in part, because alkenes are important feedstocks for producing various commercially useful materials such as detergents, high-octane gasolines, pharmaceutical products, plastics, synthetic rubbers and viscosity additives. In the commercial production of plastics, elastomers, man-made fibers, adhesives, and surface coatings, a tremendous variety of polymers are used. By far the most important industrial polymers are polymerized olefins, which comprise virtually all commodity plastics. Ethylene, a raw material in the production of polyethylene, is one of the most abundantly produced chemicals in the United States, and cost-effective methods for producing ethylene are of great commercial interest.

Olefins are unsaturated hydrocarbons (compounds containing hydrogen [H] and carbon [C]) whose molecules contain one or more pairs of carbon atoms linked together by a double bond. The olefins are classified in either or both of the following ways: (1) as cyclic or acyclic (aliphatic) olefins, in which the double bond is located between carbon atoms forming part of a cyclic (closed-ring) or an open-chain grouping, respectively; and (2) as monoolefins, diolefins, triolefins, etc., in which the number of double bonds per molecule is, respectively, one two, three, or some other number. Hence, olefins are highly desired for the production of plastics.

Generally, olefin molecules are commonly represented by the chemical formula CH₂═CHR, where C is a carbon atom, H is a hydrogen atom, and R is an atom or pendant molecular group of varying composition. The composition and structure of R determines which of the huge array of possible properties will be demonstrated by the polymer. More specifically, acyclic monoolefins have the general formula CₙH₂ₙ, where n is an integer. Acyclic monoolefins are rare in nature but are formed in large quantities during the cracking of petroleum oils to gasoline. The lower monoolefins, i.e., ethylene, propylene, and butylene, have become the basis for the extensive petrochemicals industry. Most uses of these compounds involve reactions of the double bonds with other chemical agents. Acyclic diolefins, also known as acyclic dialkenes, or acyclic dienes, with the general formula CₙH₂ₙ₋₂, contain two double bonds. They undergo reactions similar to the monoolefins. The best-known dienes are butadiene and isoprene, which are used in the manufacture of synthetic rubber.

Olefins containing two to four carbon atoms per molecule are gaseous at ordinary temperatures and pressure, and those containing five or more carbon atoms are usually liquid at ordinary temperatures. Additionally, olefins are only slightly soluble in water.

The FCC process is a catalytic thermal process, while steam cracking is a direct, non-catalytic dehydrogenation process. FCC and steam cracking are known to have drawbacks. For example, both processes are endothermic, meaning that heat energy must be supplied to drive the reaction. In addition, in FCC, coke forms on the surface of the catalyst during the cracking processes, covering active sites and deactivating the catalyst. During regeneration, the coke is burned off the catalyst to restore its activity and to provide heat needed to drive the cracking. This cycle is very stressful for the catalyst; temperatures fluctuate between extremes as coke is repeatedly deposited and burned off. Furthermore, the catalyst particles move at high speed through steel reactors and pipes, where wall contacts and interparticle contacts are impossible to avoid. The conversion of alkanes to alkenes in both FCC and steam cracking processes may be via multi reaction steps but overall reaction can be explained as a dehydrogenation reaction. One example of such a dehydrogenation reaction is the conversion of ethane to ethylene (Reaction 1):
C₂H₆+Heat→C₂H₄+H₂ (1).

FCC and steam cracking units are large and expensive because the FCC unit requires a catalyst regenerator and its catalysts use typically precious metals, as well as the steam cracking unit requiring furnaces to generate heat energy for the conversion of alkane to alkene. Recently, there has been increased interest in oxidative dehydrogenation (ODH) as an-alternative to FCC and steam cracking for the production of olefins. In ODH, alkanes are dehydrogenated in the presence of an oxidant such as molecular oxygen, typically in a short contact time reactor containing an ODH catalyst. The net ODH reaction, for example as depicted in [Reaction 2], for the conversion of ethane and oxygen to ethylene and water is:
C₂H₆+½O₂→C₂H₄+H₂O+Heat (2).
Reaction 2 may be viewed as the combination of two separate reactions: a strong exothermic combustion of alkanes [Reaction 3] and an endothermic dehydrogenation of alkanes [Reaction 4].
C₂H₆+O₂→CO₂+H₂O+Heat (3)
C₂H₆+Heat→C₂H₄+H₂ (4)

Because the exothermic combustion provides most of the heat necessary to drive the endothermic dehydrogenation reaction, ODH is a substantially autothermal process and requires no or very little energy to initiate the reaction. Energy savings over traditional, endothermal processes (FCC and steam cracking) can be significant if the heat produced with ODH is recaptured and recycled. In addition, the capital costs for olefin production via ODH are significantly less than with the traditional processes because ODH uses simple fixed bed reactor designs and high volume throughput.

Although ODH involves the use of a catalyst, which is referred to herein as an ODH catalyst, and is therefore literally a catalytic dehydrogenation, ODH is distinct from what is normally called “catalytic dehydrogenation” in that the former involves the use of an oxidant and the latter does not.

Oxidative dehydrogenation of hydrocarbons (ODH) with short contact time reactors (SCCR) is an alternative to traditional steam cracking and non-oxidative dehydrogenation processes. During an ODH reaction, an oxidant, preferably molecular oxygen, is co-fed with saturated hydrocarbons, typically one or more light hydrocarbons, optionally balanced with an inert gas, at a gas hourly space velocity (GHSV) of about 20,000 to 10,000,000 hr⁻¹. The oxidant may be fed as pure molecular oxygen, air, oxygen-enriched air, molecular oxygen mixed with a diluent gas such as nitrogen, and so forth. Oxidant in the desired amount may be added in the feed to the dehydrogenation zone. The contact time of the reactants with the catalyst is typically in the 1 to 200 ms range. The reaction pressure range is typically between 0.8 bar and 5 bars (about 80 kPa -500 kPa), and the reaction temperature is typically between 800-1,100° C.

Successful commercialization of an ODH process depends on the efficacy of the catalyst. In other words, successful commercial scale operation for catalytic hydrocarbon processing depends upon high hydrocarbon feedstock conversion at high throughput and with acceptable selectivity for the desired reaction products. In turn, the yield and selectivity of an ODH catalyst system are affected by several factors. One of the most important of these factors is the catalyst composition, which significantly affects not only the yields and product distributions but also the overall economics of the process. Unfortunately, few catalysts offer both the performance and cost necessary for economical large-scale industrial use.

Catalyst cost is one of the most significant economic considerations in ODH processes. Non-oxidative dehydrogenation reactions frequently employ relatively inexpensive iron-oxide based catalysts. In contrast, ODH catalysts typically utilize relatively expensive precious metals, e.g., platinum, as promoters that assist in the combustion reaction. In order to reduce catalyst costs, therefore, it is desirable to maximize the effectiveness of the catalyst composition, or minimize its use.

In the oxidative dehydrogenation of ethane to ethylene by the ODH process, the ODH process typically requires oxidation of a part of the ethane feed to generate the needed heat for the dehydrogenation reaction. Drawbacks of oxidizing part of the ethane feed include reduced ethylene selectivity and overall ethylene yield in the ODH process. The ODH process also typically produces combustible or reactive by-products such as methane, carbon monoxide, and the like. These by-products are typically unwanted by-products of the ODH process and are typically run to extinction.

Consequently, there is a need for a higher ethylene selectivity in the ODH process. Further needs include a higher ethane conversion. In addition, there is a need for more efficient ways to increase the ethylene selectivity and ethane conversion by the ODH process. Further needs include a more efficient use of the ODH process by-products.

### BRIEF SUMMARY OF SOME OF THE PREFERRED EMBODIMENTS

These and other needs in the art are addressed in one embodiment by an inventive method for producing olefins from light hydrocarbons. The method comprises feeding a feed to a reactor comprising a catalyst, wherein the feed comprises oxygen and a carbonaceous material comprising carbon monoxide and a light hydrocarbon; contacting the feed to the catalyst in the reactor; and converting at least a portion of the light hydrocarbon with oxygen to at least one olefin, while simultaneously converting at least a portion of the carbonaceous material with oxygen to carbon dioxide to form a product stream comprising the at least one olefin and by-products, wherein the by-products comprise at least carbon monoxide.

Another embodiment comprises a method for the production of ethylene from ethane. The method comprises feeding a reactor feed to a reactor comprising a catalyst, wherein the reactor feed comprises oxygen, carbon monoxide, and ethane; contacting the reactor feed with the catalyst; converting at least a portion of the reactor feed to form a product stream comprising ethylene; and recycling at least a portion of the product stream to the reactor.

A further embodiment comprises a method for the production of ethylene from ethane. The method comprises mixing an oxygen-containing gas and an ethane feed to form a fresh feed; combining the fresh feed with a stream comprising CO to form a reactor feed; feeding the reactor feed to a short contact time reactor containing a catalyst; contacting the reactor feed with the catalyst; converting at least a portion of the reactor feed with oxygen to form a product stream comprising ethylene and by-products, wherein the by-products include CO; separating the ethylene from the by-products to form a recycling stream and an ethylene product, wherein the recycling stream comprises CO; and sending the recycling stream comprising CO to the combining step.

Additional embodiments include a concentration of the carbon monoxide in the feed from about 1 to about 45 mole percent of the feed. Other alternative embodiments include the molar ratio of the feed to recycle at about 1:0.75 or less.

It will therefore be seen that a technical advantage of the present invention includes increasing the ethylene selectivity and ethane conversion in the oxidative conversion of ethane. Additional features and advantages of the invention include a more efficient use of the by-products produced during the oxidative conversion of ethane.

The disclosed methods comprise a combination of features and advantages, which enable the present invention to overcome the deficiencies of the prior art. The various characteristics described above, as well as other features, will be readily apparent to those skilled in the art upon reading the following detailed description and by referring to the accompanying drawings.

### BRIEF DESCRIPTION OF THE DRAWINGS

For a detailed description of the preferred embodiments of the invention, reference will now be made to the accompanying drawings in which:

FIG. 1 illustrates an ODH process with a process feed comprising a light hydrocarbon, carbon monoxide, and oxygen and having a reactor with a recycling stream comprising carbon monoxide;

FIG. 2 illustrates an ODH process having a reactor with a process feed comprising a light hydrocarbon, oxygen and carbon monoxide; and

FIG. 3 illustrates an ODH process with a process feed comprising a light hydrocarbon, and oxygen and having a reactor with a recycling stream comprising carbon monoxide.

### DETAILED DESCRIPTION OF THE PREFERRED EMBODIMENTS

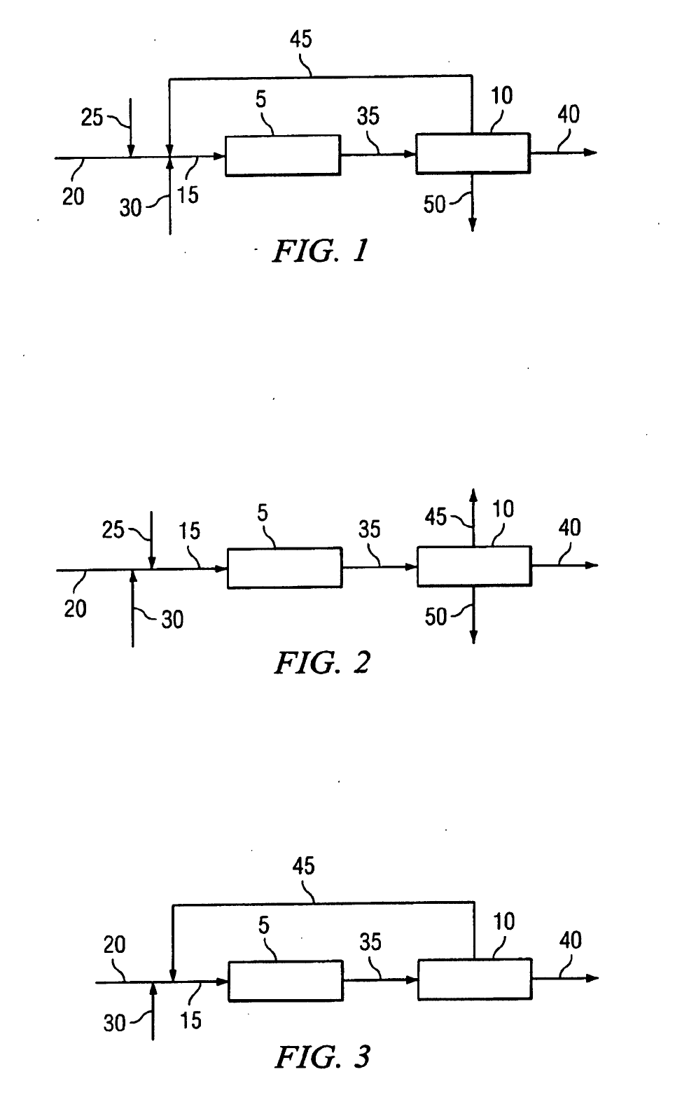

FIG. 1 illustrates an ODH process comprising an ODH reactor 5 and an olefin recovery unit 10. The ODH process further comprises a reactor feed gas 15 to ODH reactor 5, with reactor feed gas 15 comprising a light hydrocarbon feed 20, a carbon monoxide feed 25, an oxidizing feed 30, and an optional recycle product steam 45. ODH reactor 5 can comprise a short contact time reactor (SCTR), catalytic fixed bed reactor, and tube-shell reactor, preferably a SCTR. The preferred embodiments using the SCTR employ a very fast contact (e.g., millisecond range)/fast quench (e.g., less than one second) reactor assembly. Such a reactor assembly is well known in the art. For example, U.S. Pat. No. 6,409,940, which is incorporated herein by reference in its entirety to the extent that it is not contrary to the teachings of the present application, describes the use of such a reactor assembly in the production of synthesis gas from methane by catalytic partial oxidation.

ODH reactor 5 comprises at least one catalyst that is active for use in the ODH process. The catalyst may be configured in ODH reactor 5 in any suitable arrangement including fixed bed, fluidized bed, and ebulliating bed configurations, preferably fixed bed configurations. All such configurations are well known in the art.

The catalyst comprises any suitable metals that exhibit catalytic activity in the ODH process. Preferable catalysts include metals from Groups 2, 4-7, and 11-15 of the Periodic Table of Elements (according to the New Notation IUPAC Form as illustrated in, for example, the CRC Handbook of Chemistry and Physics, 82^{nd} Edition, 2001-2002; such reference being the standard herein and throughout), scandium, yttrium, actinium, iron, cobalt, nickel, their oxides and combinations thereof. The catalyst preferably includes a metal or metal oxide from Groups 2, 4-7, and 11-13 of the Periodic Table of the Elements, oxides of any such metals, or any combination thereof. More preferably, the catalyst comprises at least one of manganese, chromium, tin, copper, gold, oxides of such metals, and combinations thereof, with a loading between about 0.5 and about 20 weight percent of the catalyst, more preferably between about 1 to about 12 weight percent of the catalyst, and still more preferably between about 2 and about 10 weight percent of the catalyst.

The catalyst may further comprise promoters. Promoters are well known in the art, and the present invention may include any promoter suitable for improving the performance of the catalyst. Preferable promoters for use in the ODH process include the Groups 8, 9, and 10 metals and combinations thereof. The promoter preferably comprises platinum, palladium, iridium, rhodium, ruthenium or any combinations thereof. The promoter metal is preferably present at a promoter metal loading of between about 0.005 and about 0.20 weight percent of the catalyst, more preferably between 0.005 and 0.1 weight percent of the catalyst, still more preferably between 0.005 and 0.075 weight percent of the catalyst, and yet still more preferably between 0.005 and 0.05 weight percent of the catalyst.

In addition, in preferred embodiments, the catalyst comprises a support. Preferably, the support is selected from the group consisting of zirconia, magnesium stabilized zirconia, zirconia stabilized alumina, yttrium stabilized zirconia, calcium stabilized zirconia, alumina, titania, silica, magnesia, niobia, vanadia, nitrides, silicon nitride, carbides, silicon carbide, cordierite, cordierite-alpha alumina, zircon mullite, spodumene, alumina-silica magnesia, zircon silicate, sillimanite, magnesium silicates, zircin, petalite, carbon black, calcium oxide, barium sulfate, silica-alumina, alumina-zirconia, alumina-chromia, alumina-ceria, and combinations thereof. More preferably, the refractory support comprises alumina, zirconia, silicon nitride, magnesium oxide or combinations thereof. Suitable oxides include metastable and stable phases of the foregoing, including for example, gamma and alpha alumina and other alumina phases, any of which may be referred to as “alumina.” The support may be modified, stabilized, or pretreated in order to achieve the proper structural stability desired for sustaining the operating conditions of the catalysts made therefrom. When alumina is used as support, alumina is preferably in the form of alpha-alumina (α-alumina); however, the other forms of alumina have also demonstrated satisfactory performance.

The support can be in the shape of wire gauzes, porous monoliths, particles, and the like, preferably particles. Monoliths typically comprise any singular piece of material of continuous manufacture such as pieces of metal or metal oxide, foam materials, or honeycomb structures. The particles may comprise granules, beads, pills, pellets, cylinders, trilobes, spheres, and the like.

The term “monolith” refers to any singular piece of material of continuous manufacture such as solid pieces of metal or metal oxide or foam materials or honeycomb structures. Two or more such catalyst monoliths may be stacked in the catalyst zone of the reactor if desired. For example, the catalyst can be structured as, or supported on, a refractory oxide “honeycomb” straight channel extrudate or monolith, made of cordierite or mullite, or other configuration having longitudinal channels or passageways permitting high space velocities with a minimal pressure drop. Such configurations are known in the art and described, for example, in Structured Catalysts and Reactors, A. Cybulski and J.A. Moulijn (Eds.), Marcel Dekker, Inc., 1998, p. 599-615 (Ch. 21, X. Xu and J. A. Moulijn, “Transformation of a Structured Carrier into Structured Catalyst”), which is hereby incorporated herein by reference in its entirety to the extent that it is not contrary to the teachings of the present application.

Some preferred monolithic supports include partially stabilized zirconia (PSZ) foam (stabilized with Mg, Ca or Y), or foams of a-alumina, cordierite, titania, mullite, Zr-stabilized α-alumina, or mixtures thereof. A preferred laboratory-scale ceramic monolith support is a porous alumina foam with approximately 6,400 channels per square inch (80 pores per linear inch). Preferred foams for use in the preparation of the catalyst include those having from 30 to 150 pores per inch (12 to 60 pores per centimeter). In a preferred embodiment, the monolith is generally cylindrical with a diameter corresponding to the inside diameter of the reactor tube.

Alternatively, other refractory foam and non-foam monoliths may serve as satisfactory supports. The promoter metal precursor and any base metal precursor, with or without a ceramic oxide support forming component, may be extruded to prepare a three-dimensional form or structure such as a honeycomb, foam or other suitable tortuous-path structure.

More preferred catalyst geometries employ distinct or discrete particles. The terms “distinct” or “discrete” particles, as used herein, refer to supports in the form of divided materials such as granules, beads, pills, pellets, cylinders, trilobes, extrudates, spheres, other rounded shapes or another manufactured configuration. Alternatively, the divided material may be in the form of irregularly shaped particles. Preferably at least a majority—i.e., greater than about 50 percent—of the particles or distinct structures have a maximum characteristic length (i.e., longest dimension) equal to or less than ten millimeters, preferably less than three millimeters.

The support can be pretreated prior to application of the catalytic components (metal and promoter). The pretreatment can include heating, spray-drying to for example adjust particle sizes, dehydrating, drying, steaming and/or calcining. In the case of calcining or heating, the pretreatment step not only can stabilize the support structure but also can burn off any impurities that may contaminate the support and that may have been introduced in the support through manufacturing and/or through handling. The support is preferably pretreated by a heat treatment at a temperature between about 1,000° C. and 1,500° C. for 0.5 to 10 hours, preferably between about 2 and about 7 hours, at a heating ramp rate between 0.5 and 3° C./min (preferably at about 1.5° C./min).

The catalyst may be prepared by any suitable method known in the art. For instance, the catalyst can be prepared by incipient wetness impregnation, chemical vapor deposition, salt melt method, co-precipitation, and the like. Preferably, at least one base metal and the optional promoter metal are all deposited by the incipient wetness impregnation technique. Preferably, for a particulate-supported catalyst, the catalyst is prepared by impregnating or washcoating the metal and then loading the promoter onto the particulate support.

The following describes an exemplary application of the present invention as illustrated in FIG. 1. Light hydrocarbon feed 20, carbon monoxide feed 25, oxidizing feed 30, and optional recycle product stream 45 are mixed to form reactor feed gas 15, which is fed to ODH reactor 5. Light hydrocarbon feed 20, carbon monoxide feed 25, oxidizing feed 30, and optional recycle product stream 45 can be mixed in any order to form reactor feed gas 15. Preferably, light hydrocarbon feed 20, carbon monoxide feed 25, and oxidizing feed 30 are mixed to form a fresh feed, and the fresh feed is mixed with optional recycle product stream 45 to form reactor feed gas 15. Moreover, the constituents of reactor feed gas 15 can be mixed by any method known in the art, such as by static mixer, co-feeding into a common line, and any other suitable method. In alternative embodiments, light hydrocarbon feed 20, carbon monoxide feed 25, oxidizing feed 30 and optional recycle product stream 45 are not all mixed together and at least one of such feeds is fed independently to ODH reactor 5 and/or at least a portion of at least one of such feeds is fed independently to ODH reactor 5. Light hydrocarbon feed 20 comprises one or more alkanes having between two and ten carbon atoms. Preferably, light hydrocarbon feed 20 comprises one or more alkanes having between two and five carbon atoms, such as ethane, propane, butane, isobutane, pentane, and the like. Most preferably, light hydrocarbon feed 20 comprises ethane. Carbon monoxide feed 25 comprises carbon monoxide gas. Optional recycle product stream 45 preferably comprises at least carbon monoxide. Optional recycle product stream 45 may further comprise hydrogen.

Oxidizing feed 30 comprises any suitable gas capable of oxidizing at least a portion of light hydrocarbon feed 20, at least a portion of carbon monoxide feed 25, and at least a portion of recycle product stream 45. Preferably, oxidizing feed 30 comprises an oxygen-containing gas. Examples of suitable oxygen-containing gases include pure oxygen gas, oxygen gas mixed with a diluent gas such as nitrogen, air, and O₂-enriched air.

The ratio of oxygen atoms in oxidizing feed 30 to carbon atoms in light hydrocarbon feed 20 can be any suitable ratio sufficient to achieve desired selectivity and conversion. Preferably, the atomic O:C ratio is 5:1 or less. More preferably, the atomic O:C ratio is 3.5:1 or less. Still more preferably, the atomic O:C ratio is between 0.4:1 and 3.5:1. In other embodiments, the atomic O:C ratio is 2:1 or less. Thus, the molar ratio of O₂ to light hydrocarbon in reactor feed gas 15 is preferably 5:1 or less; more preferably 3.5:1 or less; still more preferably between 0.4:1 and 3.5:1; and alternatively 2:1 or less. In addition, the concentration of carbon monoxide in reactor feed gas 15 comprises any suitable concentration to achieve desired olefin selectivity and hydrocarbon conversion, with at least a portion of the carbon monoxide used in lieu of at least a portion of the light hydrocarbons to be oxidized (by combustion). Preferably, the concentration of carbon monoxide in reactor feed gas 15 is from about 1 to about 45 mole percent. More preferably, the concentration of carbon monoxide in reactor feed gas 15 is from about 5 to about 35 mole percent. In alternative embodiments (not illustrated), other combustible gases such as hydrogen, lower alkanes, and the like can be fed to ODH reactor 5 either independently or each may be mixed with reactor feed gas 15.

Reactor feed gas 15 (or just light hydrocarbon feed 20) can be preheated before being fed into ODH reactor 5. Methods for heating gas are well known in the art, and reactor feed gas 15 or light hydrocarbon feed 20 can be preheated by any suitable method, such as electric heater, fire heater, heat exchanger, and the like. Preferably, reactor feed gas 15 or light hydrocarbon feed 20 is preheated by fire heater. Reactor feed gas 15 or light hydrocarbon feed 20 can be preheated to any desired temperature suitable for the ODH process. Preferably, reactor feed gas 15 or light hydrocarbon feed 20 is preheated to about 600° C. or less. More preferably, reactor feed gas 15 or light hydrocarbon feed 20 is preheated to about 450° C. or less. Most preferably, reactor feed gas 15 or light hydrocarbon feed 20 is preheated to about 300° C. or less. In alternative embodiments, reactor feed gas 15 and light hydrocarbon feed 20 are not preheated before being fed to ODH reactor 5. In other alternative embodiments, reactor feed gas 15 and/or at least one of light hydrocarbon feed 20, carbon monoxide feed 25, oxidizing feed 30, and any other feeds are preheated before being fed to ODH reactor 5. In further alternative embodiments, fresh feed is preheated.

ODH reactor 5 operates at any conditions sufficient for the oxidative dehydrogenation of the alkane. For instance, ODH reactor 5 preferably operates at pressures of about 500 psig or less. The pressure is more preferably between 4 psig and about 300 psig. In some alternative embodiments, the pressure is between 45 psig and about 125 psig. The gas hourly space velocity (GHSV) of ODH reactor 5 is preferably from about 20,000 hr⁻¹ to about 10,000,000 hr⁻¹, and more preferably from about 50,000 hr⁻¹ to about 4,000,000 hr⁻¹. In addition, the reaction temperature in ODH reactor 5 is preferably from about 600° C. to about 2,000° C., more preferably from about 700° C. to about 1,500° C., and most preferably from about 800° C. to about 1,200° C. It is to be understood that reactor operating conditions and other parameters such as catalyst composition and reactant mixture composition can be controlled in any desired manner in which the desired ODH process is favored.

ODH reactor product 35 comprises the converted olefin product. Olefin products can comprise any desired olefins. The preferable olefin product comprises ethylene. It is to be understood that the reactor conditions and other parameters can be controlled to obtain any desired alkane conversion and olefin selectivity. Preferred alkane conversion is at least about 40 percent, and the olefin selectivity is at least about 30 percent. More preferably, the alkane conversion is at least about 60 percent, and the olefin selectivity is at least about 50 percent. Still more preferably, the alkane conversion is at least about 65 percent, and the olefin selectivity is at least about 55 percent. Most preferably, the alkane conversion is at least about 70 percent, and the olefin selectivity is at least about 60 percent.

ODH reactor product 35 also comprises by-products such as combustible or reactive reaction products and non-reactive by-products. To separate the olefin product from the by-products. ODH reactor product 35 is introduced to olefin recovery unit 10. It is understood that methods and equipment for separating olefins are well known in the art. Olefin recovery unit 10 may comprise any such suitable methods and equipment for separating the olefin product from the by-products in ODH reactor product 35, such as cryogenic separation, distillation, selective membrane, combinations thereof, and any other suitable method and equipment. Olefin recovery unit 10 preferably uses cryogenic separation to separate the olefin product but may also employ other techniques.

Olefin recovery unit 10 separates ODH reactor product 35 into olefin product 40, recycle product stream 45 comprising reactive products, and non-reactive products 50. Olefin product 40 comprises at least a portion of the olefins in ODH reactor product 35, preferably at least about 90 percent of the olefins in ODH reactor product 35. Non-reactive products 50 comprise at least a portion of the non-reactive by-products in ODH reactor product 35, preferably at least about 90 percent of the non-reactive by-products in ODH reactor product 35. The non-reactive by-products comprise water, carbon dioxide, and the like.

Recycle product stream 45 comprises at least a portion of the combustible or reactive reaction products in ODH reactor product 35, preferably at least about 50 percent of the combustible or reactive reaction products in ODH reactor product 35. The combustible or reactive reaction products comprise by-products such as ethane, carbon monoxide, hydrogen, and the like.

Recycle product stream 45 can have any molecular weight as dictated by the ODH process, preferably a molecular weight of about 30 or less. Preferably, recycle product stream 45 is recycled and mixed into reactor feed gas 15. Recycle product stream 45 can also be preheated before being fed into ODH reactor 5, either separately and/or with reactor feed gas 15 or light hydrocarbon feed 20. In alternative embodiments, at least a portion of recycle product stream 45 can be recycled and fed separately to ODH reactor 5 from reactor feed gas 15. At least a portion of reactive products comprised in recycle product stream 45 can be oxidized in ODH reactor 5 in place of a portion of the light hydrocarbon. Therefore, the conversion of the light hydrocarbon and olefin selectivity can be increased. It is to be understood that the reactor conditions and other parameters such as reactor feed, which comprises light hydrocarbon feed 20, carbon monoxide feed 25, and/or oxidizing feed 30, can be controlled when recycle product stream 45 is recycled to ODH reactor 5. In a preferred embodiment, all of recycle product stream 45 is recycled to ODH reactor 5. The molar ratio of recycle product stream 45 to fresh feed can be any desired ratio effective for the ODH process. Preferably, the molar ratio of fresh feed to recycle product stream 45 is about 1:0.75 or less and more preferably 1:0.5 or less. In alternative embodiments, at least a portion of recycle product stream 45 is recycled to ODH reactor 5. In other alternative embodiments (not illustrated), none of the recycle product stream 45 is recycled to ODH reactor 5.

FIG. 2 illustrates an ODH process similar to FIG. 1 except that reactor feed gas 15 does not comprise recycle product stream 45, which is not recycled to ODH reactor 5. The carbon monoxide content in reactor feed gas 15 is provided by carbon monoxide feed 25.

FIG. 3 illustrates an ODH process similar to FIG. 1 except the carbon monoxide content in reactor feed gas 15 is provided solely by the recycling of carbon monoxide (formed in ODH reactor 5) via recycle product stream 45 (which comprises CO) from olefin recovery unit 10 downstream of ODH reactor 5.

To further illustrate various illustrative embodiments of the present invention, the following examples are provided.

TABLE I

| | Mole % | % | % | % | % | % |
|---|---|---|---|---|---|---|
| | CO in the | Ethane | Acetylene | Propylene | Ethylene | Methane |
| Example | feed | Conversion | Sel. | Sel. | Sel. | Sel. |
| 1 | 43 | 74.7 | 2.2 | 0.0 | 74.7 | 5.7 |
| 2 | 33 | 72.3 | 2.0 | 0.2 | 79.4 | 5.7 |
| 3 | 22 | 70.7 | 1.7 | 0.3 | 79.1 | 5.3 |
| 4 | 12 | 68.9 | 1.4 | 0.3 | 77.6 | 5.0 |
| 5 | 0 | 55.9 | 0.3 | 0.7 | 71.8 | 4.1 |

### EXAMPLES 1-5

Ethane, oxygen, carbon monoxide, and nitrogen were supplied from gas cylinders and controlled by respective gas flow controllers. Such gases were mixed through a static mixer and then fed to the reactor. The reactor comprised a 9/16 inch (14.3 mm) inside diameter quartz tubing. The catalysts comprised 0.4 g of a 0.1Pd-0.4Cu/2.4Mn/ZrO2 catalyst. The catalysts were packed between two blank ceramic foams, with one blank ceramic foam as the catalyst bed support and the other as a shield. An electric heater was located upstream of the catalyst bed and was used to pre-heat the reactor feed to about 300° C. A cooler was located downstream of the catalyst bed and was used to cool the product stream to below about 35° C., which condensated most of the water vapor. A small part of the gas product stream from the reactor was sent to a gas chromatograph for composition analysis.

The catalyst, 0.1Pd-0.4Cu/2.4Mn/ZrO₂, was made using an incipient wetness impregnation technique, which is well known in the art. The zirconia support material (ZrO₂) used in the catalyst example was purchased from Süd-Chemie (Louisville, Ky.) and was comprised of particles of about 0.84-1.19 mm (16-20 mesh) size. For preparation by the incipient wetness impregnation technique, manganese nitrate [Mn(NO₃)₂], copper nitrate [Cu(NO₃)₂], and palladium nitrate [Pd(NO₃)₂] were used, with all purchased from Aldrich (Milwaukee, Wis.). A solution of manganese nitrate was first applied to the support particles. After the base metal was applied, the catalyst precursor was dried at 125° C. for 1 hour followed by calcination in air at 500° C. for 3 hours. A solution comprising both copper nitrate and palladium nitrate was applied, dried and calcined using the same procedures as described for manganese. The amounts of palladium, copper, and manganese in the impregnation solutions were selected such that the catalyst comprised 0.1 g Pd, 0.4 g Cu , and 2.4 g Mn per 100 g of ZrO₂. Before testing, the finished catalyst was then reduced in an atmosphere with an equimolar mixture of hydrogen and nitrogen at 125° C. for 0.5 hour and then at 500° C. for 3 hours.

The total feed flow rate to the reactor was about 3 standard liters per minute (SLPM), which corresponds to a gas hourly space velocity of about 354,000 hr⁻¹. The ethane flow rate was about 1.08 SLPM, and the oxygen flow rate was about 0.52 SLPM. Therefore, the inlet ethane to oxygen molar ratio was about 2.08. The carbon monoxide in the reactor feed was varied from about 0 to about 43.0 mole percent. Nitrogen was used as a balance gas to maintain the total gas flow rate. The operating pressure of the reactor was about 4.0 psig (129 kPa).

The results as listed in Table I indicate that the ethane conversion increased from 55.9 percent to 72.3 percent with an increasing carbon monoxide concentration in the feed from 0 to 33.0 mole percent, and the ethylene selectivity correspondingly increased from 71.8 percent to 79.4 percent. A further increase in carbon monoxide concentration to 43.0 percent resulted in a further increase of ethane conversion but a decrease in ethylene selectivity. However, as indicated in Table I, the ethylene selectivity with carbon monoxide feed was higher than with no carbon monoxide feed. Consequently, the addition of carbon monoxide to the reactor resulted in an improvement in ethane conversion and ethylene selectivity.

It is to be further understood that the present invention is not limited to carbon monoxide feed 25. In alternative embodiments (not illustrated), when reactive products 45 are recycled and fed to ODH reactor 5, carbon monoxide feed 25 can be substantially shut off, with reactive products 45 comprising substantially all of the carbon monoxide feed to ODH reactor 5. In other alternative embodiments (not illustrated), at least one balance gas can be fed to ODH reactor 5 to maintain a total gas flow to ODH reactor 5. The balance gas can comprise nitrogen, helium, argon, and the like. The balance gas can be mixed into reactor feed gas 15 or fed separately to ODH reactor 5. The balance gas is fed to ODH reactor 5 to maintain a total gas flow rate to ODH reactor 5.

Although the present invention and its advantages have been described in detail, it should be understood that various changes, substitutions, and alterations may be made herein without departing from the spirit and scope of the invention as defined by the appended claims.

1. A method for producing olefins from light hydrocarbons, the method comprising:
(A) feeding a feed to a reactor comprising a catalyst, wherein the feed comprises oxygen and a carbonaceous material comprising carbon monoxide and a light hydrocarbon;
(B) contacting the feed to the catalyst in the reactor; and
(C) converting at least a portion of the light hydrocarbon with oxygen to at least one olefin, while simultaneously converting at least a portion of the carbonaceous material with oxygen to carbon dioxide to form a product stream comprising the at least one olefin and by-products, wherein the by-products comprise at least carbon monoxide.

2. The method of claim 1, wherein the feed has a molar ratio of oxygen to light hydrocarbon of about 3.5:1 or less.

3. The method of claim 1, wherein the feed has a molar ratio of oxygen to light hydrocarbon between 0.4:1 and 3.5:1.

4. The method of claim 1, wherein the feed has a carbon monoxide concentration from about 1 to about 45 mole percent.

5. The method of claim 1, wherein the feed has a carbon monoxide concentration from about 5 to about 35 mole percent.

6. The method of claim 1, wherein the feed further comprises hydrogen.

7. The method of claim 1, wherein the light hydrocarbon comprises one or more alkanes having between two and ten carbon atoms.

8. The method of claim 1, wherein the light hydrocarbon comprises one or more alkanes having between two and five carbon atoms.

9. The method of claim 1, wherein the light hydrocarbon comprises ethane.

10. The method of claim 1, further comprising mixing an oxygen feed, a carbon monoxide feed, and a light hydrocarbon feed to form the feed to the reactor.

11. The method of claim 1, wherein the catalyst comprises at least one metal selected from the group consisting of manganese, chromium, tin, copper, gold, oxides of such metals, and combinations thereof.

12. The method of claim 1, wherein the catalyst comprises a promoter selected from the group consisting of platinum, palladium, iridium, rhodium, ruthenium, and combinations thereof.

13. The method of claim 1, wherein the catalyst comprises a support selected from the group consisting of alumina, zirconia, silicon nitride, magnesium oxide, and combinations thereof.

14. The method of claim 1, wherein the feed is preheated before being fed to the reactor.

15. The method of claim 14, wherein the feed is preheated to about 600° C. or less.

16. The method of claim 14, wherein the feed is preheated to about 450° C. or less.

17. The method of claim 14, wherein the feed is preheated to about 300° C. or less.

18. The method of claim 1, wherein the reactor operates at pressures of about 500 psig or less.

19. The method of claim 1, wherein the reactor operates at pressures between about 4 psig and about 300 psig.

20. The method of claim 1, wherein the reactor operates at a GHSV from about 20,000 hr⁻¹ to about 10,000,000 hr⁻¹.

21. The method of claim 1, wherein the step (C) occurs in the reactor at a gas temperature from about 700° C. to about 1,500° C.

22. The method of claim 1, wherein step (C) further comprises producing the at least one olefin with a light hydrocarbon conversion of at least about 40 percent, and an olefin selectivity of at least about 30 percent.

23. The method of claim 1, wherein step (C) further comprises producing the at least one olefin with a light hydrocarbon conversion of at least about 60 percent, and an olefin selectivity of at least about 50 percent.

24. The method of claim 1, wherein step (C) further comprises producing the at least one olefin with a light hydrocarbon conversion of at least about 65 percent, and an olefin selectivity of at least about 55 percent.

25. The method of claim 1, wherein step (C) further comprises producing the at least one olefin with a light hydrocarbon conversion of at least about 70 percent, and an olefin selectivity of at least about 60 percent.

26. The method of claim 1, wherein step (C) further comprises separating the at least one olefin from the by-products to form an olefin product.

27. The method of claim 26, wherein the at least one olefin is separated from the by-products by cryogenic separation.

28. The method of claim 1, wherein at least a portion of the product stream is recycled to the reactor.

29. The method of claim 28, wherein the molar ratio of the fresh feed to the product stream recycle is about 1:0.75 or less.

30. A method for the production of ethylene from ethane, the method comprising:
(A) feeding a reactor feed to a reactor comprising a catalyst, wherein the reactor feed comprises oxygen, carbon monoxide, and ethane;
(B) contacting the reactor feed with the catalyst;
(C) converting at least a portion of the reactor feed to form a product stream comprising ethylene; and
(D) recycling at least a portion of the product stream to the reactor.

31. The method of claim 30, further comprising passing the product stream through a separation unit to form an ethylene-enriched product and at least one CO-enriched by-product stream.

32. The method of claim 31, wherein step (D) comprises recycling the at least one CO-enriched by-product stream to the reactor.

33. The method of claim 30, wherein the concentration of the carbon monoxide in the reactor feed comprises from about 1 to about 45 mole percent.

34. The method of claim 30, wherein the reactor feed has a molar ratio of oxygen to ethane of about 3.5:1 or less.

35. A method for the production of ethylene from ethane, the method comprising:
(A) mixing an oxygen-containing gas and an ethane feed to form a fresh feed;
(B) combining the fresh feed with a stream comprising CO to form a reactor feed;
(C) feeding the reactor feed to a short contact time reactor containing a catalyst;
(D) contacting the reactor feed with the catalyst;
(E) converting at least a portion of the reactor feed with oxygen to form a product stream comprising ethylene and by-products, wherein the by-products include CO;
(F) separating the ethylene from the by-products to form a recycling stream and an ethylene product, wherein the recycling stream comprises CO; and
(G) sending the recycling stream comprising CO to step (B).

36. The method of claim 35, wherein the reactor feed has a molar ratio of oxygen to light hydrocarbon of about 3.5:1 or less.

37. The method of claim 35, wherein the reactor feed has a molar ratio of oxygen to light hydrocarbon between 0.4:1 and 3.5:1.

38. The method of claim 35, wherein the concentration of the carbon monoxide in the reactor feed comprises from about 1 to about 45 mole percent.

39. The method of claim 35, wherein the reactor feed is preheated before being fed to the reactor.

40. The method of claim 35, wherein the reactor operates at a GHSV from about 20,000 hr⁻to about 10,000,000 hr⁻.
